SEQUENCE LISTING (9 sequences)

SEQ ID NO: 1 - protein, 108 aa, synthetic construct
  REGION 1..108: note = Synthetic peptide, VC VR1
SELTQDPAVSVALGQTVRITCQGDSLRSSYASWYRQRPGQAPVPVIYGKNNRPSGIPDRFSGSSSGNTASLTITGAQAEDEADYYCXSSYAWLPYVVFGGGTKLTVLG

SEQ ID NO: 2 - protein, 117 aa, synthetic construct
  REGION 1..117: note = Synthetic peptide, HC VR2
EVQLAESGGGLVQPGGSLRLSCAASGFTFSSDAMSWVRQAPGKGLEWVSAISGSGGSTYYADSVKGRFTISRDNSKNTLYLQMNSLRAEDTAVYYCAKSNEFLFDYWGQGTLVTVSR

SEQ ID NO: 3 - protein, 106 aa, synthetic construct
  REGION 1..106: note = Synthetic peptide, LC CR3
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 4 - protein, 330 aa, synthetic construct [sequence omitted]

SEQ ID NO: 5 - protein, 330 aa, synthetic construct
  REGION 1..330: note = Synthetic peptide, HC CR5
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 106 aa, synthetic construct
  REGION 1..106: note = Synthetic peptide, LC CR6
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 7 - protein, 330 aa, synthetic construct
  REGION 1..330: note = Synthetic peptide, LC CR7
  VARIANT 180: note = Xaa can be any naturally occurring amino acid
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYXSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 106 aa, synthetic construct
  REGION 1..106: note = Synthetic peptide, HC CR8
  VARIANT 6: note = Xaa can be any naturally occurring amino acid
  VARIANT 8: note = Xaa can be any naturally occurring amino acid
  VARIANT 46: note = Xaa can be any naturally occurring amino acid
  VARIANT 57: note = Xaa can be any naturally occurring amino acid
GQPKAXPXVTLFPPSSEELQANKATLVCLISDFYPGAVTVAWKADXSPVKAGVETTXPSKQSNNKYAASSYLSLTPEQWKSHRSYSCQVTHEGSTVEKTVAPTECS

SEQ ID NO: 9 - protein, 327 aa, synthetic construct
  REGION 1..327: note = Synthtic peptide, HC CR9
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2025/0340633 A1
(43) Pub. Date: Nov. 6, 2025

(54) HUMAN ANTIBODIES AND USES THEREOF

(71) Applicant: DIATHEVA S.R.L., Cartoceto (IT)

(72) Inventors: Valentina FIORI, Fano (IT); Sabrina DOMINICI, Fermignano (IT); Michela CENTONZE, Fano (IT)

(21) Appl. No.: 18/724,143

(22) PCT Filed: Dec. 29, 2022
(86) PCT No.: PCT/EP2022/087993

§ 371 Date: Jun. 26, 2024

(30) Foreign Application Priority Data
Dec. 29, 2021 (IT) 102021000033002

(51) Int. Cl. C07K 16/28; A61K 47/68; A61K 49/00; A61K 51/10; A61P 35/00

(52) U.S. Cl. CPC C07K 16/2803; A61K 47/6853; A61K 49/0002; A61K 51/1048; A61P 35/00; A61K 2121/00; A61K 2123/00; C07K 2317/21

(57) ABSTRACT

It forms an object of the present invention a fully human antibody specific for CEACAM1, comprising the light chain variable region sequences defined by SEQ ID NO: 1, the heavy chain variable region sequences defined by SEQ ID NO: 2 and the human antibody Immunoglobulin G class I constant region sequences. It forms a further object of the present invention the use of said antibody for cancer diagnosis and/or therapy.

### CROSS-REFERENCE TO RELATED APPLICATIONS

This application is the U.S. national phase of International Application No. PCT/EP2022/087993 filed Dec. 29, 2022, which designated the U.S. and claims priority to IT 102021000033002 filed Dec. 29, 2021, the entire contents of each of which are hereby incorporated by reference.

### REFERENCE TO SEQUENCE LISTING SUBMITTED ELECTRONICALLY

The content of the electronically submitted sequence listing (Name: 2149-338_Sequence Listing.xml, Size: 16,072 bytes, and Date of Creation: Jan. 14, 2025) is herein incorporated by reference in its entirety.

The present invention relates to antibodies binding with high affinity to Carcinoembryonic antigen (CEA) and CEACAM1 (CEA Cell Adhesion Molecule 1).

### BACKGROUND

Conventional pharmacological approaches for the therapy of tumours suffer from poor selectivity, thus compromising dose escalation to therapeutically active levels. Therefore, the development of selective, and better tolerated, cancer therapeutics represents an important goal in the research of new and more effective treatment. One way to improve the selectivity of therapeutic molecules is to target the tumour site, thereby sparing normal tissues. Antibody-based cancer treatments offer a solution to this problem and have given promising results in several malignancies (Goydel RS, Rader C Antibody-based cancer therapy. Oncogene 2021, 40:3655-3664).

Carcinoembryonic antigen is an attractive target for immunotherapeutic purposes because of its expression profile, its role in tumour progression, and its immunogenicity. CEA belongs to the CD66 immunoglobulin superfamily that also comprises CEACAM1. CEACAM1 was found to be involved in progression and metastatic potential of melanoma and lung cancer. CEACAM1 re-expression often occurs in the advanced stages of many malignancies such as bladder cancer, thyroid cancer, gastric carcinoma, pancreatic cancer, metastatic colon cancer and multiple myeloma. (Fiori V, et al. Ann Ist Super Sanita 2012, 48:161-71). The involvement of CEACAM1 in cancer progression can be partially explained by its central role in the angiogenic switch of tumours. CEACAM1 is involved in the switch from non-invasive and non-vascularised to invasive and vascularised bladder cancer (Oliveira-Ferrer L, et al. Dual role of carcinoembryonic antigen-related cell adhesion molecule 1 in angiogenesis and invasion of human urinary bladder cancer. Cancer Res. 2004, 15:8932-8). In addition, CEACAM1 is emerging as a novel immune checkpoint target involved in the inhibition of antitumor immune responses (Dankner M, et al. CEACAM1 as a multi-purpose target for cancer immunotherapy. Oncoimmunology 2017, 6:7). WO2011160859A1 describes a single chain variable fragment specific for each of carcinoembryonic antigen (CEA) and CEA related Cell Adhesion Molecule 1 (CEACAM1), scFvDIATHIS-1.

EP3909601 describes anti CEACAMs antibodies, demonstrating same properties in term of dose-response curve, kinetics, and Kd determination when the CDRs were in mouse monoclonal IgG antibodies or in human IgG1, IgG2a or IgG4 antibodies.

An important need persists for a mean to target in a selective and efficient manner CEA and CEACAM1 to gain the full benefit of these therapeutic agents.

### DESCRIPTION

### Description of the Figures

FIG. 1: Creation of IgG1 and IgG4 anti-CEACAM1 mAbs stable producing clones in HEK293 cells. (A) IgG1 pool selection by ELISA assay, black column: selected pool; (B) IgG1 clone selection by ELISA assay, black column: selected clone; (C) IgG1 subclone selection by ELISA assay, black column: selected subclone; (D) IgG4 pool selection by ELISA assay, black column: selected pool; (E) IgG4 clone selection by ELISA assay, black column: selected clone.

FIG. 2: SEC-HPLC analysis of (A) IgG1, clone 12 and (B) IgG4, clone 2 anti-CEACAM1 mAbs.

FIG. 3: SDS-PAGE of IgG1 anti-CEACAM1 mAb clone 12.3.

FIG. 4: ELISA titration assays showing CEACAM1 recognition by IgG1 (black) and IgG4 (grey) purified after a transient transfection in HEK293T cells.

FIG. 5: ELISA titration assays showing CEACAM1 recognition by (A) IgG1 clone 12 (black line, according to the invention) and IgG4 clone 2 (grey line, comparative); (B) scFvDIATHIS-1 (comparative).

FIG. 6: IgG1 and IgG4 reactivity on CEACAM1 antigen expressed on metastatic melanoma cells by Flow cytometry. MelC cells were reacted with 50, 25 or 12 μg/ml of IgG1 or IgG4. Negative control (CTR-) is represented by MelC cells reacted with secondary antibodies only. (A) Cytograms display the results obtained with the indicated antibodies. (B) Mean Fluorescence values representing the affinity of the binding

FIG. 7: IgG1 and IgG4 reactivity on CEACAM1 antigen expressed on metastatic melanoma cells by Flow cytometry. MelP5 cells were reacted with 50, 25 or 12 μg/ml of IgG1 or IgG4 anti-CEACAM1 mAb. Negative control is represented by MelP5 cells reacted with secondary antibodies only (CTR-). (A) Cytograms display the results obtained with the indicated antibodies. (B) Mean Fluorescence values representing the affinity of the binding

FIG. 8: Analysis of IgG1 clone 12, IgG4 clone 2 (comparative), scFv (comparative) reactivity by Flow cytometry, mean fluorescence values, on CEACAM1 and CEA antigens expressed on (A) bladder cancer cells; (B) colorectal cancer cells.

FIG. 9: Analysis of IgG1 purified from the stable clone 12.3, reactivity on CEACAM1 and CEA antigens expressed on metastatic bladder cancer cells or colorectal cancer cells by Flow cytometry. Cells were reacted with 50, 20, 10 or 5 μg/ml of DIA-12.3 IgG1. Negative control represents cells reacted with secondary antibodies only. (A) Cytograms display the results obtained with the indicated antibody concentrations. (B) Mean Fluorescence values, representing the affinity of the binding.

FIG. 10: ELISA titration assays showing CEACAM1 recognition by IgG1 (grey) and IgG1 N297A mutated (black).

FIG. 11: LDH assay (A) on MelC target cells co-incubated with IL-2 activated NK-92 effector cells at the indicated ratio, after incubation with DIA-12.3 Ab at the indicated concentration. (B) on MelC, TCCSUP, 5637, HT29, HN target cells, respectively, co-incubated with IL-2 activated NK-92 effector cells at E:T ratio of 10:1, after incubation with 20 μg/ml DIA-12.3 Ab.

FIG. 12: DIA-12.3 IgG1 reactivity on CEACAM1 antigen expressed on neutrophils by Flow cytometry. Neutrophils were reacted with 10 μg/ml of DIA-12.3 IgG1 mAb. Negative control is represented by cells reacted with secondary antibodies only (Anti-human Fc antibody). Cytograms display the results obtained with the indicated antibodies.

FIG. 13: FITC Annexin V+PI staining on neutrophils, exposed or not to DIA-12.3 IgG1. (A) % live cells; (B) % early proapoptotic cells; (C) late apoptotic cells.

FIG. 14: DIA-12.3 IgG1 and DIA-12.3 N297A IgG1 reactivity on CEACAM1 antigen expressed on MelC by Flow cytometry. Binding percentages are indicated in the graphs.

### DETAILED DESCRIPTION

It forms an object of the present invention a composition comprising a fully human antibody specific for CEA and CEACAM1, comprising the light chain variable region sequence defined by SEQ ID NO: 1 (LCVR1); the heavy chain variable region sequence defined by SEQ ID NO: 2 (HCVR2) and the human antibody Immunoglobulin G constant region sequences; the light chain variable region sequence being: SELTQDPAVSVALGQTVRITCQGDSLRSSYASWYRQRPGQAPVPVIY GKNNRPSGIPDRFSGSSSGNTASLTITGAQAEDEADYYCX6SSYAWL PYVVFGGGTKLTVLG (SEQ ID NO: 1) wherein X6 is any naturally occurring amino acid. In an embodiment, X6 is N or Q or A or L. In a preferred embodiment, X6 is Q.

The heavy chain variable region sequence being:

(SEQ ID NO: 2)
EVQLAESGGGLVQPGGSLRLSCAASGFTFSSDAMSWVRQAPGKGLE
WVSAISGSGGSTYYADSVKGRFTISRDNSKNTLYLQMNSLRAEDT
AVYYCAKSNEFLFDYWGQGTLVTVSR.

In an embodiment, the light chain constant region sequences are defined by SEQ ID NO: 8, GQPKAX1PX2VTLFPPSSEELQANKATLVCLISDFYPGAVTVAWKADX 3SPVKAGVETTX4PSKQSNNKYAASSYLSLTPEQWKSHRSYSCQVTH EGSTVEKTVAPTECS, wherein X1, X2, X3, X4 are any naturally occurring amino acid.

In an embodiment, X1 is N or A, X2 is T or S, X3 is G or S, X4 is K or T. In an embodiment, the heavy chain constant region sequences are defined by SEQ ID NO: 7, ASTKGPSVFPLAPSSKSTSGGTAALGCLVKDYFPEPVTVSWNSGALT SGVHTFPAVLQSSGLYSLSSVVTVPSSSLGTQTYICNVNHKPSNTKVD KKVEPKSCDKTHTCPPCPAPELLGGPSVFLFPPKPKDTLMISRTPEVT CVVVDVSHEDPEVKFNWYVDGVEVHNAKTKPREEQYXSSTYRVVSV LTVLHQDWLNGKEYKCKVSNKALPAPIEKTISKAKGQPREPQVYTLPP SRDELTKNQVSLTCLVKGFYPSDIAVEWESNGQPENNYKTTPPVLDS DGSFFLYSKLTVDKSRWQQGNVFSCSVMHEALHNHYTQKSLSLSPG K, wherein X5 any naturally occurring amino acid

In an embodiment, X5 is N or A.

In an embodiment, X1 is N, X2 is T, X3 is G, X4 is K.

In an alternative embodiment, X1 is A, X2 is S, X3 is S, X4 is T.

In an embodiment, the light chain variable region sequence is LCVR1, X6 being N, the heavy chain variable region sequence is HCVR2, the light chain constant region sequence is defined by SEQ ID NO: 3; the heavy chain constant region sequence is defined by SEQ ID NO: 4, wherein SEQ ID NO: 3 is GQPKANPTVTLFPPSSEELQANKATLVCLISDFYPGAVTVAWKADGS PVKAGVETTKPSKQSNNKYAASSYLSLTPEQWKSHRSYSCQVTHEG STVEKTVAPTECS and SEQ ID NO: 4 is ASTKGPSVFPLAPSSKSTSGGTAALGCLVKDYFPEPVTVSWNSGALT SGVHTFPAVLQSSGLYSLSSVVTVPSSSLGTQTYICNVNHKPSNTKVD KKVEPKSCDKTHTCPPCPAPELLGGPSVFLFPPKPKDTLMISRTPEVT CVVVDVSHEDPEVKFNWYVDGVEVHNAKTKPREEQYNSTYRVVSVL TVLHQDWLNGKEYKCKVSNKALPAPIEKTISKAKGQPREPQVYTLPPS RDELTKNQVSLTCLVKGFYPSDIAVEWESNGQPENNYKTTPPVLDSD GSFFLYSKLTVDKSRWQQGNVFSCSVMHEALHNHYTQKSLSLSPGK.

In an embodiment, the light chain variable region sequence is LCVR1, X6 being Q, the heavy chain variable region sequence is HCVR2, the light chain constant region sequence is defined by SEQ ID NO: 3; the heavy chain constant region sequence is defined by SEQ ID NO: 4.

In an embodiment, the light chain variable region sequence is LCVR1, X6 being N, the heavy chain variable region sequence is HCVR2, the light chain constant region sequence is defined by SEQ ID NO: 3; the heavy chain constant region sequence is defined by SEQ ID NO: 5, wherein SEQ ID NO: 5 is ASTKGPSVFPLAPSSKSTSGGTAALGCLVKDYFPEPVTVSWNSGALT SGVHTFPAVLQSSGLYSLSSVVTVPSSSLGTQTYICNVNHKPSNTKVD KKVEPKSCDKTHTCPPCPAPELLGGPSVFLFPPKPKDTLMISRTPEVT CVVVDVSHEDPEVKFNWYVDGVEVHNAKTKPREEQYASTYRVVSVL TVLHQDWLNGKEYKCKVSNKALPAPIEKTISKAKGQPREPQVYTLPPS RDELTKNQVSLTCLVKGFYPSDIAVEWESNGQPENNYKTTPPVLDSD GSFFLYSKLTVDKSRWQQGNVFSCSVMHEALHNHYTQKSLSLSPGK.

In an embodiment, the light chain variable region sequence is LCVR1, X6 being Q, the heavy chain variable region sequence is HCVR2, the light chain constant region sequence is defined by SEQ ID NO: 3; the heavy chain constant region sequence is defined by SEQ ID NO: 5.

In an embodiment, the light chain variable region sequence is LCVR1, X6 being N, the heavy chain variable region sequence is HCVR2, the light chain constant region sequence is defined by SEQ ID NO: 6; the heavy chain constant region sequence is defined by SEQ ID NO: 4, wherein SEQ ID NO: 6 is GQPKAAPSVTLFPPSSEELQANKATLVCLISDFYPGAVTVAWKADSSP VKAGVETTTPSKQSNNKYAASSYLSLTPEQWKSHRSYSCQVTHEGS TVEKTVAPTECS.

In an embodiment, the light chain variable region sequence is LCVR1, X6 being Q, the heavy chain variable region sequence is HCVR2, the light chain constant region sequence is defined by SEQ ID NO: 6; the heavy chain constant region sequence is defined by SEQ ID NO: 4.

In an embodiment, the heavy chain constant region sequence is defined by SEQ ID NO: 9, wherein SEQ ID NO: 9 is ASTKGPSVFPLAPCSRSTSESTAALGCLVKDYFPEPVTVSWNSGALT SGVHTFPAVLQSSGLYSLSSVVTVPSSSLGTKTYTCNVDHKPSNTKV DKRVESKYGPPCPPCPAPEFLGGPSVFLFPPKPKDTLMISRTPEVTCV VVDVSQEDPEVQFNWYVDGVEVHNAKTKPREEQFNSTYRVVSVLTV LHQDWLNGKEYKCKVSNKGLPSSIEKTISKAKGQPREPQVYTLPPSQ EEMTKNQVSLTCLVKGFYPSDIAVEWESNGQPENNYKTTPPVLDSDG SFFLYSRLTVDKSRWQEGNVFSCSVMHEALHNHYTQKSLSLSLGK.

In an embodiment, the human Immunoglobulin G is Class I or Class IV IgG where the class IV IgG is stabilized with the S228P mutation, reference is made to SEQ ID NO: 9. In a preferred embodiment, said human Immunoglobulin G is Class I.

In an embodiment, said antibody is DIA-12.3 IgG1, wherein the light chain variable region sequence is SEQ ID NO: 1, X6 being N, the heavy chain variable region sequence is SEQ ID NO: 2, the light chain constant region sequence is SEQ ID NO: 3; the heavy chain constant region sequence is SEQ ID NO: 4.

In an embodiment, said antibody is DIA-12.3 IgG1 N87A, wherein the light chain variable region sequence is SEQ ID NO: 1, X6 being A, the heavy chain variable region sequence is SEQ ID NO: 2, the light chain constant region sequence is SEQ ID NO: 3; the heavy chain constant region sequence is SEQ ID NO: 4.

In an embodiment, said antibody is DIA-12.3 IgG1 N297A, wherein the light chain variable region sequence is SEQ ID NO: 1, the heavy chain variable region sequence is SEQ ID NO: 2, the light chain constant region sequence is SEQ ID NO: 3; the heavy chain constant region sequence is SEQ ID NO: 5.

n an embodiment, said antibody is DIA-12.3 IgG1 N87A, N297A wherein the light chain variable region sequence is SEQ ID NO: 1, X6 being A, the heavy chain variable region sequence is SEQ ID NO: 2, the light chain constant region sequence is SEQ ID NO: 3; the heavy chain constant region sequence is SEQ ID NO: 5.

The DIA-12.3 IgG1 N297A is a particularly preferred embodiment, wherein the introduced mutation at the glycosylation site N297, known to reduce effector function, does not impact the affinity of the antibody for the target.

In an embodiment, said antibody is DIA-2.2(3) IgG4, wherein the light chain variable region sequence is LCVR1, the heavy chain variable region sequence is HCVR2, the light chain constant region sequence is defined by SEQ ID NO: 3; the heavy chain constant region sequence is defined by SEQ ID NO: 9.

In an embodiment, said antibody is DIA-2.2 IgG4, wherein the light chain variable region sequence is LCVR1, the heavy chain variable region sequence is HCVR2, the light chain constant region sequence is defined by SEQ ID NO: 6; the heavy chain constant region sequence is defined by SEQ ID NO:9.

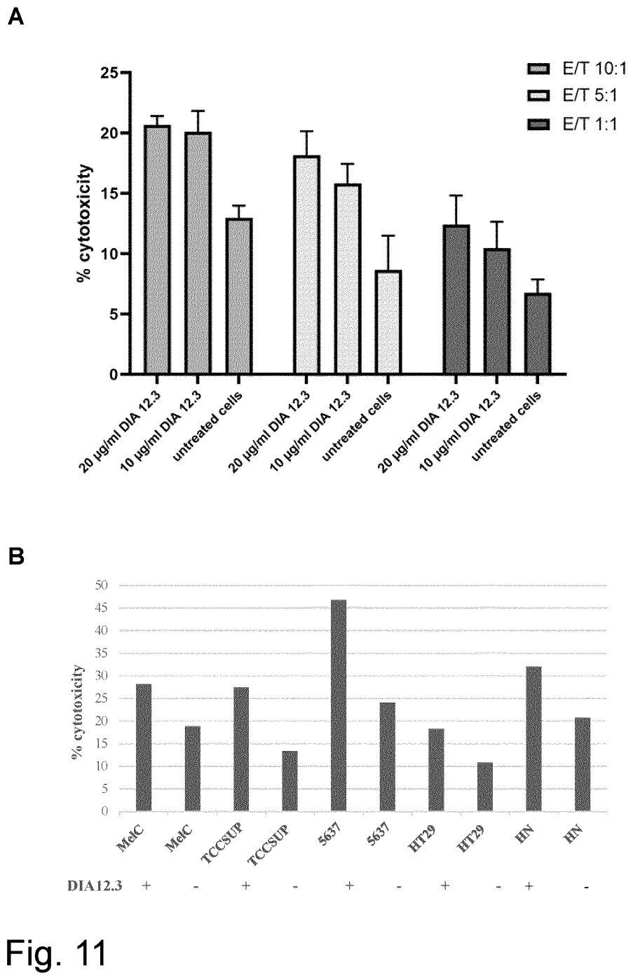

The antibody according to the present invention demonstrated capable to enhances NK cell mediated cytotoxicity against tumor cells. Data reported in FIG. 11 demonstrates the impact of DIA-12.3 IgG1 on Melanoma cells (panel A) and on bladder, colorectal and head and neck cancer cells (panel B).

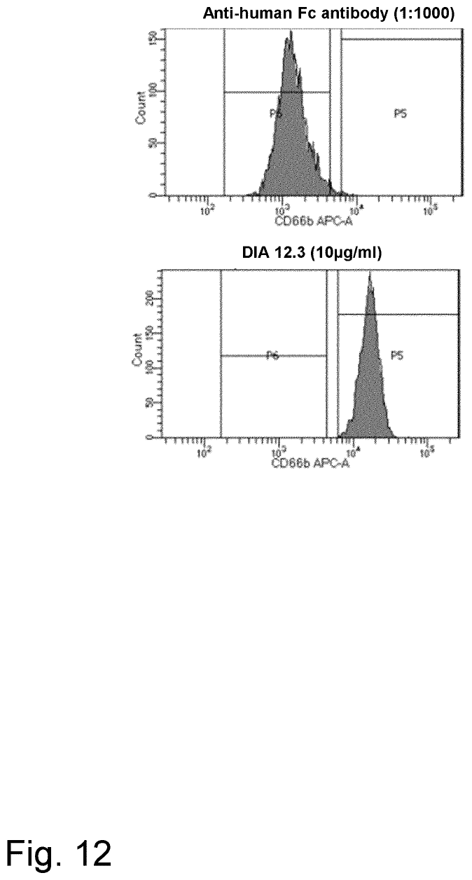

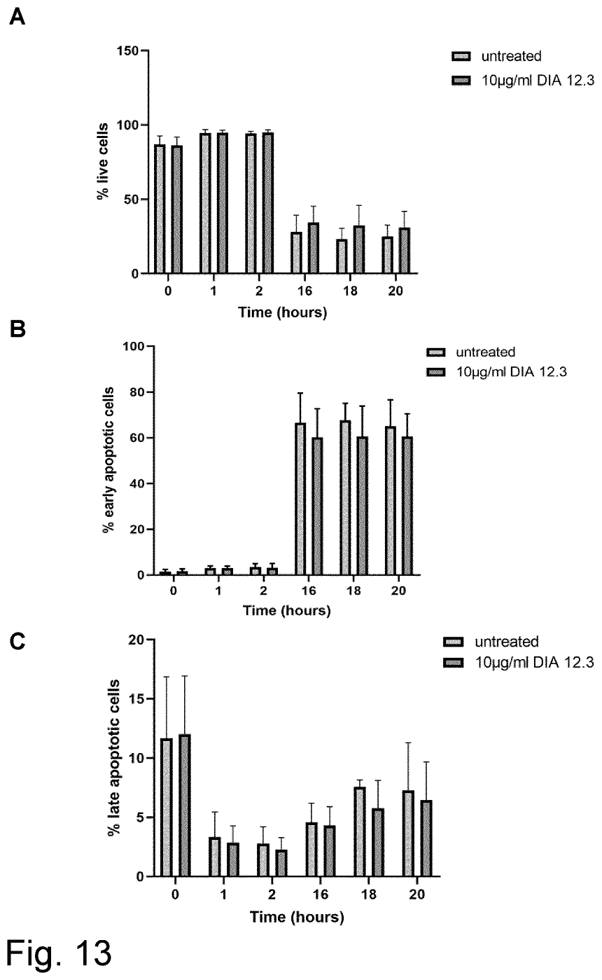

Moreover, DIA-12.3 IgG1 binds CEACAM1 antigen, when expressed on neutrophils isolated from healthy donors, as shown in FIG. 12, without exerting any toxicity on the same cells, FIG. 13.

In another embodiment, the antibody specific for CEA and CEACAM1 according to the present invention is conjugated to at least one diagnostic and/or therapeutic agent.

Non limiting examples of said at least one therapeutic agent are an antibody, an antigen-binding antibody fragment, a cytotoxic agent, a drug, a toxin, a radionuclide, boron atoms, an immunomodulator, a photoactive therapeutic agent, an immunoconjugate, an oligonucleotide and a hormone.

Non limiting examples of said at least one diagnostic agent are a radionuclide, a radiological contrast agent, a paramagnetic ion, a metal, a fluorescent label, a chemiluminescent label, an ultrasound contrast agent and a photoactive agent.

In another embodiment, the antibody specific for CEA and CEACAM1 according to the present invention is combined with at least one diagnostic and/or therapeutic agent.

In an embodiment, it is here claimed a pharmaceutical composition comprising at least one antibody according to the present invention.

In an embodiment, said pharmaceutical composition further comprises an additional therapeutic and/or diagnostic agent. In yet another embodiment, a method for diagnosing or treating a patient comprises the step of administering in an appropriate regimen the conjugate or the combination of the previous preferred embodiments.

In yet another embodiment, said method comprises the step of administering the pharmaceutical composition according to the present invention in combination with conventional tumor therapy, wherein the conventional tumor therapy is selected from the group consisting of: radiotherapy, chemotherapy, antibody therapy, and surgical tumor removal.

In yet another embodiment, the antibody specific for CEA and CEACAM1, conjugates or compositions thereof is claimed for use in a method for the treatment of a pathologies selected in the group comprising: medullary thyroid cancer (MTC), colorectal cancer, hepatocellular carcinoma, liver cancer, gastric cancer, oesophageal cancer, lung cancer, non-small cell lung cancer, breast cancer, pancreatic cancer, ovarian cancer, uterine cancer, cervical cancer, endometrial cancer, head-and-neck cancer, bladder cancer, urothelial cancer, prostate cancer, hematopoietic cancer, leukaemia or melanoma. In yet another embodiment, the antibody specific for CEA and CEACAM1, conjugates or compositions thereof is claimed for use in a method for the diagnosis of cancer.

Another preferred embodiment comprises the DNA sequence of the heavy and light chain sequences described above.

Other objects, features and advantages of the present invention will become apparent from the appended claims.

In a particularly preferred embodiment of the present invention, the light chain variable region sequence defined by SEQ ID NO: 1 (LCVR1); the heavy chain variable region sequence defined by SEQ ID NO: 2 (HCVR2) are grafted into a fully human antibody.

In this context, human antibody refers to any antibody that occurs in a human or an engineered antibody that has been designed, in some respect, to be compatible with the human immune system.

Particularly preferred for this purpose are antibodies that, broadly, do not elicit an adverse immune response in a patient.

### Advantages

The authors of the present invention have surprisingly found IgG antibodies which bind with high efficiency and in a highly selective manner CEA and CEACAM1. The here described IgG antibodies are surprisingly better than the already available scFv.

In a preferred embodiment, the IgG is a Class I IgG. Surprisingly, this embodiment has been demonstrated to have a higher affinity than the embodiment where the IgG is a Class IV IgG.

In a further preferred embodiment, the IgG is a Class I IgG and a mutation has been introduced at the conserved amino acid N297. In this preferred embodiment, the effector function is reduced, and the affinity is maintained.

Surprisingly, this is further improved in the embodiment wherein a mutation is introduced in the light chain variable region sequence, a position 87. Preferably, the conserved amino acid N87 is substituted with a Glutamine, or with an Alanine, or with a Leucine. To note, there are no indication in the state of the art to introduce mutation in the variable region sequence.

The examples that follows have not limiting scope, wherein the scope of protection of the present invention is defined by the claims.

### EXPERIMENTAL SECTION

### Example 1: Generation of Monoclonal IgG1 and IgG4 Antibodies Transient Production of IgG1 and IgG4 Antibodies in HEK 293T Cells

HEK293T and HEK293 (Human Embryonic Kidney) cells were purchased from DSMZ (Germany). HEK293T cells were kept in culture in DMEM medium (Dulbecco's Modified Eagle Medium) (Carlo Erba, Italy), supplemented with 10% decomplemented foetal bovine serum (FBS, Sigma Aldrich, USA), 2 mM L-glutamine (Sigma Aldrich, USA). All cell lines were grown in T-Flask (VWR, USA) and incubated at 37° C. in an incubator with 5% CO₂ in a humidified atmosphere. HEK293T cells were transiently co-transfected with plasmids encoding the Heavy chain (HC) and the Light chain (LC) of the antibodies containing the same variable regions as the scFv DIATHIS-1 fused to the IgG1 or IgG4 with the S228P mutation subclasses constant regions. Briefly, the day before the transfection 2×10⁶ HEK293T cells in 10 ml of media were seeded in each of 20 T75-flask. The day after, the cells were co-transfected with a mixture of HC and LC plasmids, total of 10 μg of plasmid DNA and the transfection reagent GeneCellin (Eurobio, France). Cells from 10 flasks were co-transfected with HC and LC plasmids belonging to the IgG1 subclass and cells from 10 flasks with HC and LC plasmids belonging to the IgG4 subclass.

Transfected cells were grown for 7 days at 37° C. in CO₂ incubator and the cell culture medium was then collected for antibodies purification with protein A affinity chromatography. The yield of purified antibody was similar but higher for the IgG1 compared to the IgG4 (1.52 mg vs 1.25 mg).

### Selection of Stable Clones for the Production of IgG1 and IgG4

For the selection of a stable cell line expressing the IgG1 or the IgG4(S228P) antibodies, a bicistronic vector containing both the HC and LC genes was created for each antibody. The expression vector used contains the gene for the Neomycin resistance allowing the use of this antibiotic for the selection of stable clones.

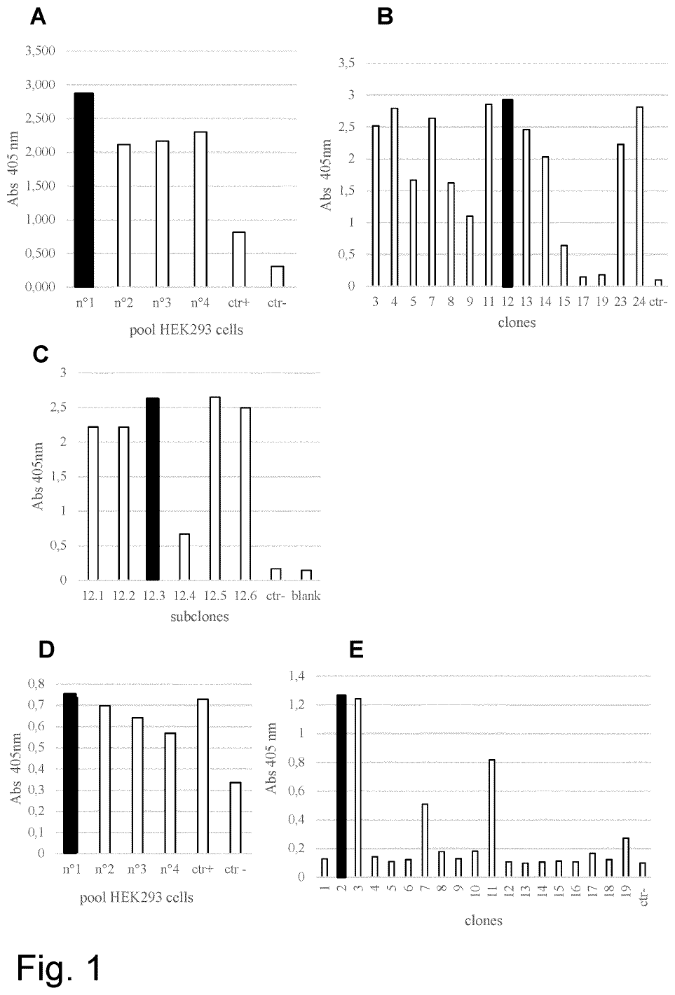

HEK293 cells were used for the stable pools generation and for clones selection. Briefly, HEK293 cells were transfected with the IgG1 or IgG4(S228P) bicistronic plasmid. The plasmid was linearized prior the transfection. Two days after the transfection, cells were expanded in medium containing neomycin (G418) at a concentration of 500 μg/ml. The cells were fed with the selective media every 3-4 days. After 14 days of selection, neomycin resistant pools were tested for antibody expression analyzing the reactivity by ELISA against the CEACAM1 antigen. 100 μl of culture medium from each pool were analyzed in duplicate with the method described below. FIG. 1A is exemplificative of the expression levels in IgG1 transfected pools, and FIG. 1D of IgG4 transfected pools. The best expressing pools were used for cloning through limiting dilution in 96 well plates. Selected clones were screened for IgG1 or IgG4 expression in the culture media by ELISA.

As shown in FIG. 1, IgG1 pools and clones have a much higher reactivity compared to the IgG4 counterparts. IgG1 clone 12 (FIG. 1B) and IgG4 clone 2 (FIG. 1E) that showed the highest reactivity against CEACAM1, were expanded for antibodies production. Briefly, the cells were seeded in T-182 flasks at the density of 250.000 cells/ml. Cells were grown for 2 days at 30° C. followed by further 7 days at 37° C. in CO₂ incubator and the culture media harvested and used for antibodies purification with protein A affinity chromatography. The yield values were 9.2 mg/L for IgG1 clone 12 and 4.6 mg/L for IgG4 clone 2.

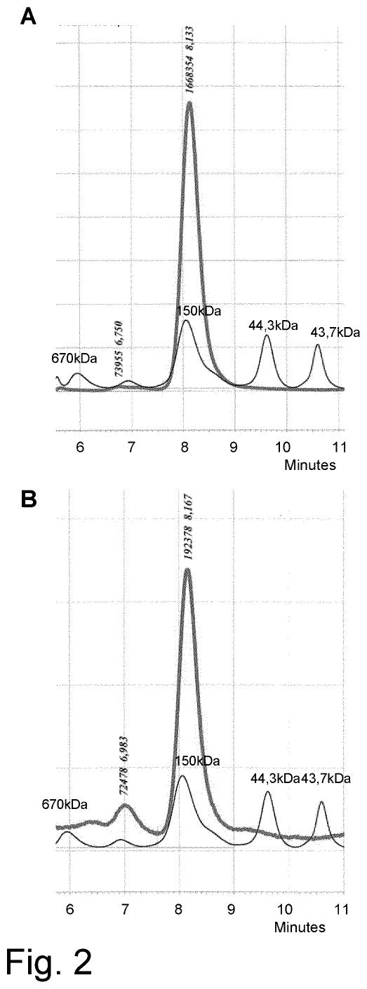

In SEC-HPLC analysis performed under isocratic conditions, both IgG1 purified clone 12 and IgG4 purified clone 2 eluted as a single peak with a molecular weight of about 150 kDa corresponding to the monomeric form of IgG (FIG. 2), confirming the integrity of the antibodies. Exclusion chromatography was performed using the TSKgel G3000SWXL column (Tosoh Bioscience, Japan), with eluent consisting of 0.1 mol/L Na₂SO₄+0.05% NaN₃ in 0.1 mol/L phosphate buffer (pH=6.7). Molecular weights were calculated based on a standard curve obtained by calibrating the system with molecules of known molecular weight: Thyroglobulin 670 kDa, Y-globulin 150 kDa, Ovalbumin 44.3 kDa, Ribonuclease A 43.7 kDa, Para-aminobenzoic acid 137.1 Da.

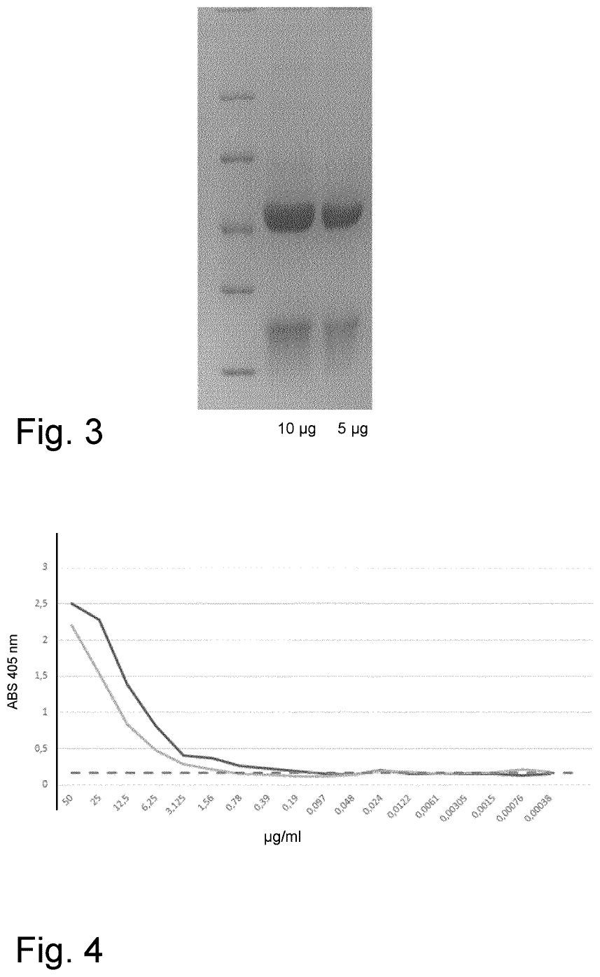

IgG1 clone 12 was used for a further subcloning step with limiting dilution in 96 well plates. Results are showed in FIG. 1C. Among the subclones arised from the limiting dilution, the 12.3 showing the highest reactivity with CEACAM1 was expanded and used for antibody production as described above. The yield of purified mAb was 10.4 mg/L. SDS-PAGE, 10% acrylamide, showed the purity of the antibody (FIG. 3).

### Example 2: ELISA Titration

Purified antibodies were analysed for their reactivity against CEACAM1 antigen in ELISA titration assay as follow:

96 wells microtiter plates (MAXISORB NUNC) were coated with 100 μl/well of CEACAM1 antigen (Diatheva) solution 1 μg/ml diluted in PBS pH 7.3 and incubated overnight (ON) at 37° C. Plates were washed five times with PBS added with 0.05% (v/v) Tween 20 pH 7.3 (PBST) (the same washing procedure was repeated after each incubation step during the assays) and then blocked with 1% (w/v) BSA dissolved in PBS (150 μl/well) and maintained at 37° C. for 60 min. After washing, serial dilutions of IgG1 or IgG4 in PBS containing 2% (w/v) not-fat dry milk (PBSM) were added to the plate (100 μl/well) and incubated for 90 min at 37° C. After washes, plates were incubated at 37° C. for 60 min with an anti-human Fc antibody HRP conjugated (Meridian) diluted 1:500 v/v in PBSM. Finally, the detection was performed after addition of 2,2′-azino-bis(3-ethylbenzthiazoline-6-sulphonic acid) (ABTS; Roche, 10102946001) as substrate. The absorbance values were read by a microplate reader (Bio-Rad Laboratories) at 405 nm after 45 min. In FIG. 4 are reported the results obtained with IgG1 (black line) and IgG4 (grey line) antibodies purified after transient expression in HEK293T cells. The absorbance of the mock sample is shown by the dotted line. The ELISA assay highlighted a higher affinity for the CEACAM1 antigen of the antibody belonging to the IgG1 subclass compared to the same antibody with the IgG4 (S228P) constant region. The EC50 were 12.79 and 26.71 μg/ml for the IgG1 and the IgG4, respectively.

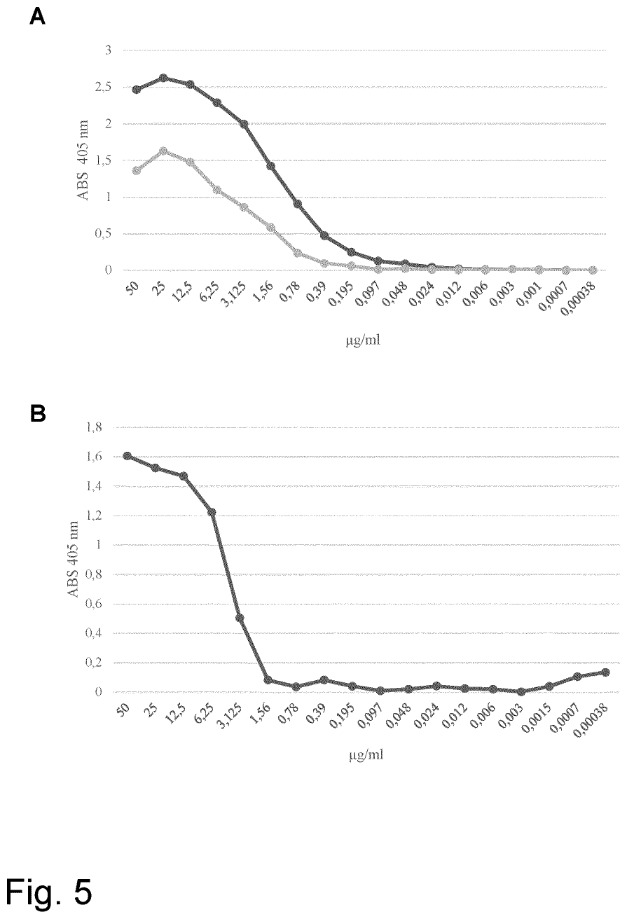

ELISA assay was then performed with antibodies isolate from stable clones: IgG1 clone 12 and IgG4 clone 2 and, for comparative purpose only, to detect scFvDIATHIS-1 reactivity (as described in WO2011160859A1). In this case, the plates were incubated at 37° C. for 60 min with an anti-His6 monoclonal antibody (100 μl/well; AbD Serotec, MCA1396) diluted 1:1,000 (v/v) in PBSM. Then, a goat anti-mouse polyclonal antibody HRP conjugated (Bio-Rad, 172-1011EDU) diluted 1:1,000 v/v in PBSM was dispensed (100 μl/well) and incubated for 60 min at 37° C. Results shown on FIG. 5, panel A, confirmed a higher affinity for the IgG1 (EC50 1.27 μg/ml) compared to the IgG4 (EC50 2.554 μg/ml). In addition, both IgG1 and IgG4 have a higher reactivity in ELISA compared to scFvDIATHIS-1 (FIG. 5 panel B) that showed an EC50 of 4.155 μg/ml, i.e., doubled compared to the IgG4 and 4 fold that of the IgG1.

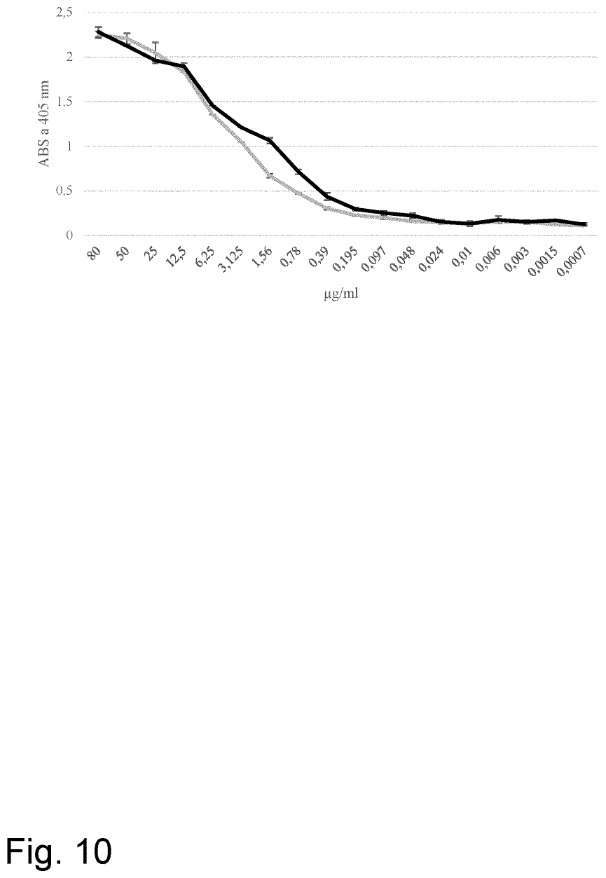

A further experiment has been performed wherein the IgG1 subclone 12.3 (i.e., DIA-12.3 IgG1) antibody has been tested by ELISA together with the N297A mutant (i.e. DIA-12.3 IgG1 N297A). Results are shown in FIG. 10. The N297A mutated antibody (black line) shows an overlapping performance than is naïve counterpart (grey line).

### Example 3: Flow Cytometry Assay on Target Cells

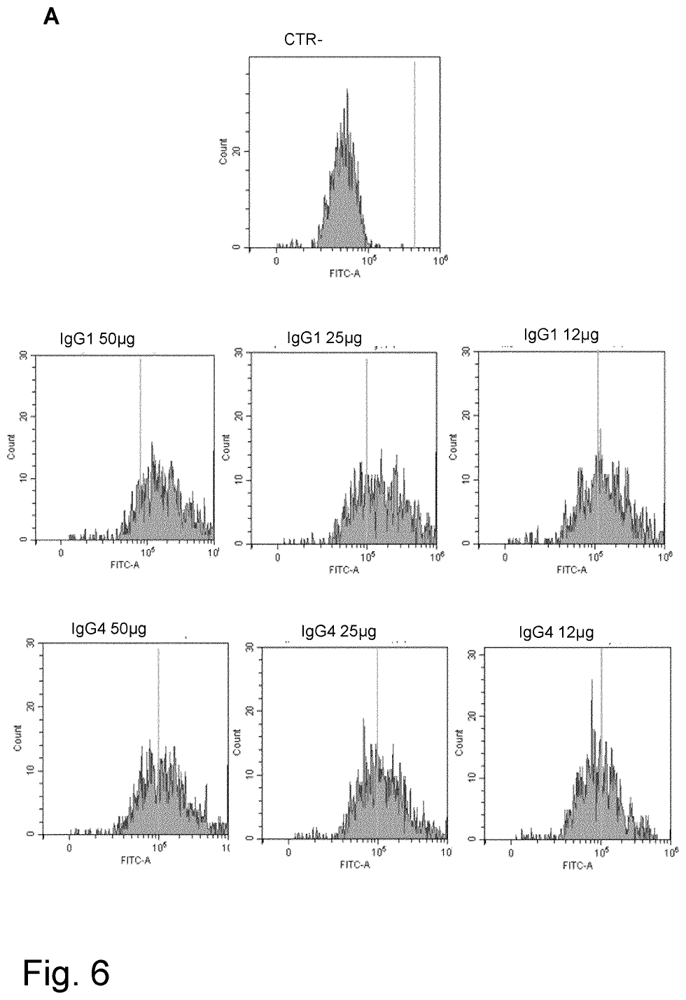

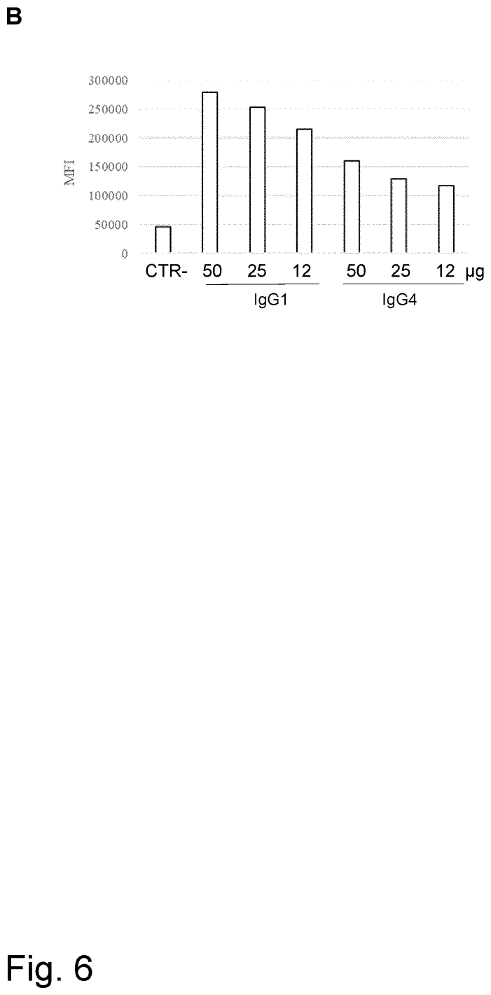

The antibodies purified after transient expression in HEK293T cells, were also evaluated for the binding of CEACAM1 expressed on the surface of metastatic melanoma cells MelC and MelP5. The analysis is carried out by dispensing 5×10⁵ MelC cells (primary metastatic melanoma cells) or MEL-P5 melanoma cell lines in 1 mL of medium per tube. The cells are then washed twice in PBS by centrifuging at 1,600 rpm for 6 min and finally resuspended in 200 μl of a blocking solution consisting of 1×PBS and 1% w/v BSA and incubated for 30 minutes at room temperature (RT). After this phase, the antibody diluted in 200 μl of block solution is added and incubated for 60 minutes at room temperature. At the end of the incubation, two washes are carried out in PBS and each cell pellet is resuspended in 200 μl of a solution consisting of an anti-human-FITC antibody diluted 1:1000 v/v in blocking solution, the incubation is maintained for 60 minutes at RT. Cells are then washed once with PBS and centrifuged as above, the supernatant is removed, and pellet resuspended in 500 μl of PBS. The samples were subjected to flow cytometric analysis with FacScan (Becton Dickinson). In a first assay, MelC cells were reacted with 50, 25 or 12 μg/ml of anti CEACAM1 IgG1 or IgG4. Results are reported in FIG. 6.

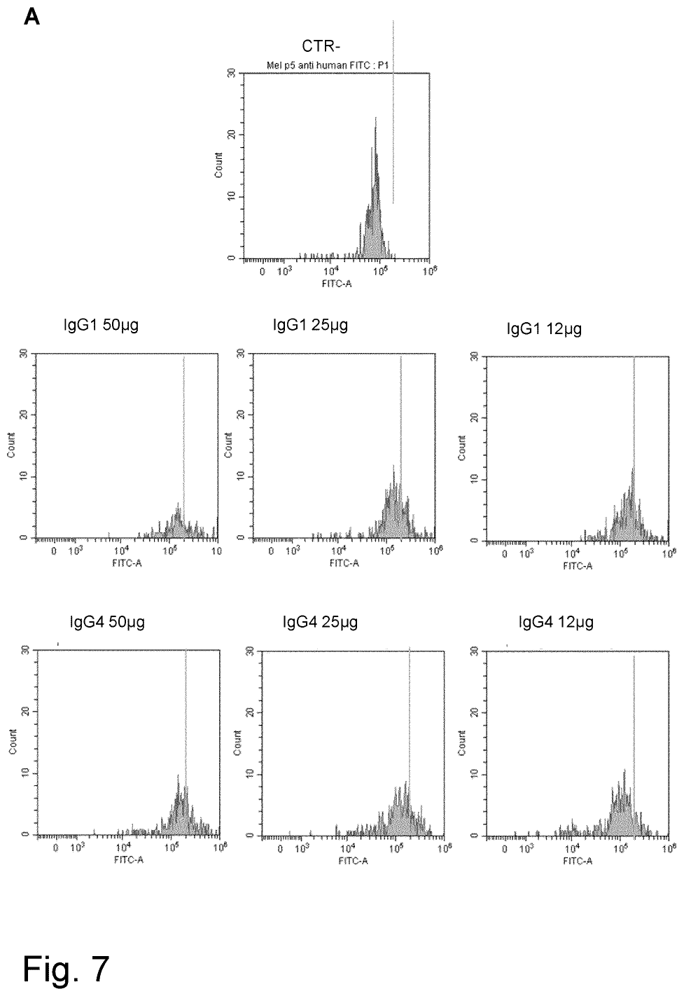

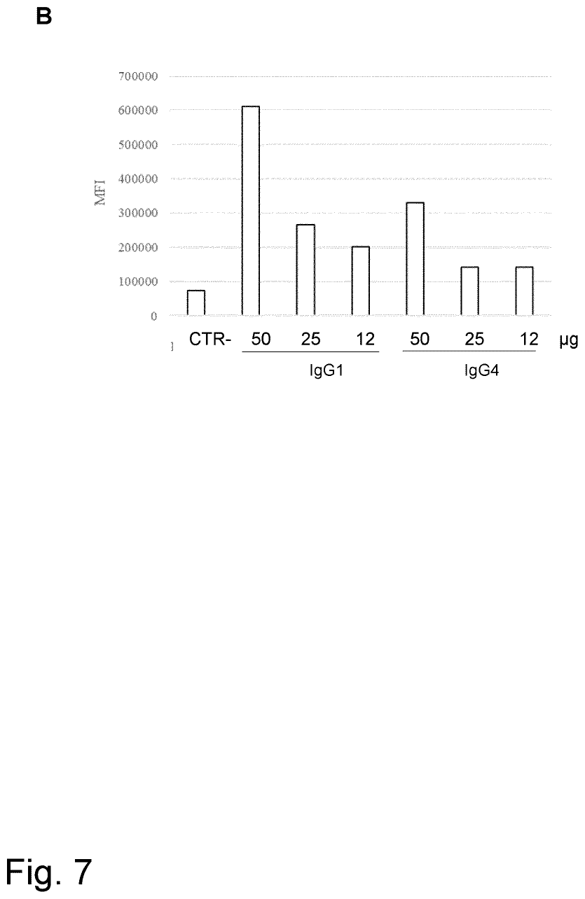

The assay was repeated on a different cell line, the human melanoma cell line MEL-P5. Results are reported in FIG. 7.

On the two cell lines, the percentage of positive cells as well as the values of the mean fluorescence intensity (MFI), representing a measure of the antibody affinity, were higher for the IgG1 compared to the IgG4 at every concentration tested confirming the results obtained in ELISA.

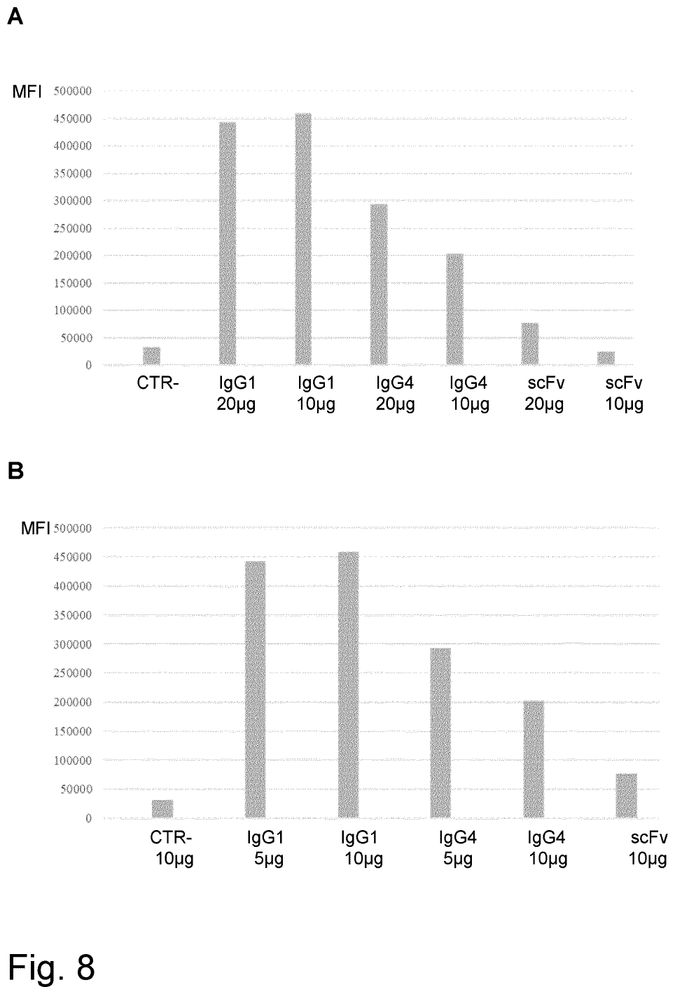

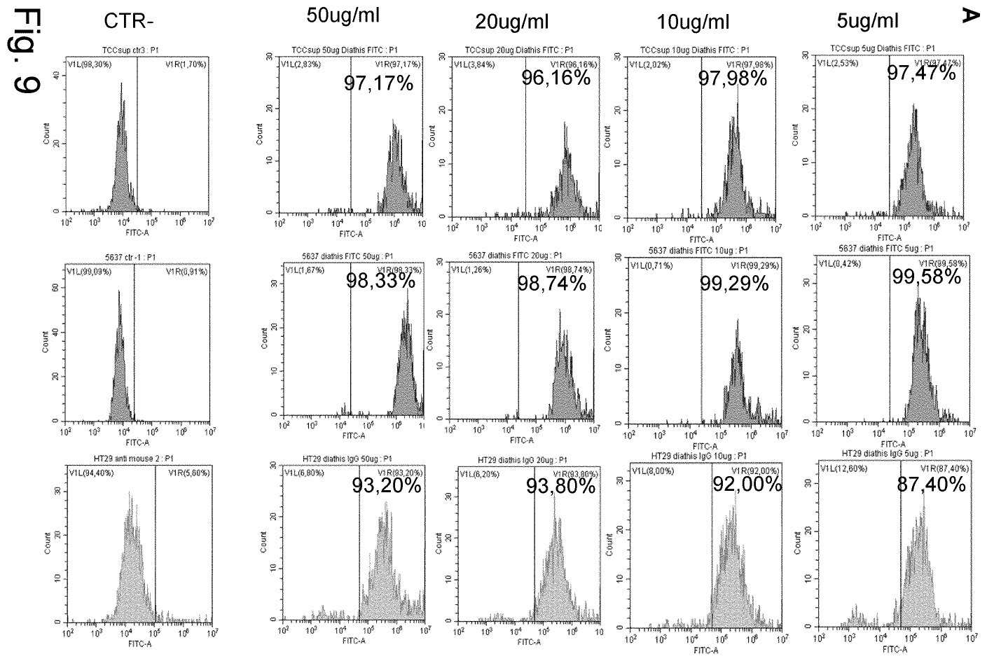

In a third assay, HT29 colorectal cancer cells and TCCSUP bladder cancer cells were reacted with 20 or 10 μg/ml of IgG1 clone 12 or IgG4 clone 2 and, for comparative purposes, scFv DIATHIS-1. Results of the MFI are reported in FIG. 8, with reference to bladder cancer cells, panel A, and with reference to colorectal cancer cells, panel B. In the two experimental setting, i.e., antibody obtained from transiently expressing cells or from stable clones, it is evident that the affinity obtained with the IgG1 antibody and IgG4 antibody according to the present invention is higher with respect to the scFv. IgG1 is confirmed by this assay, too, as the most preferred embodiment.

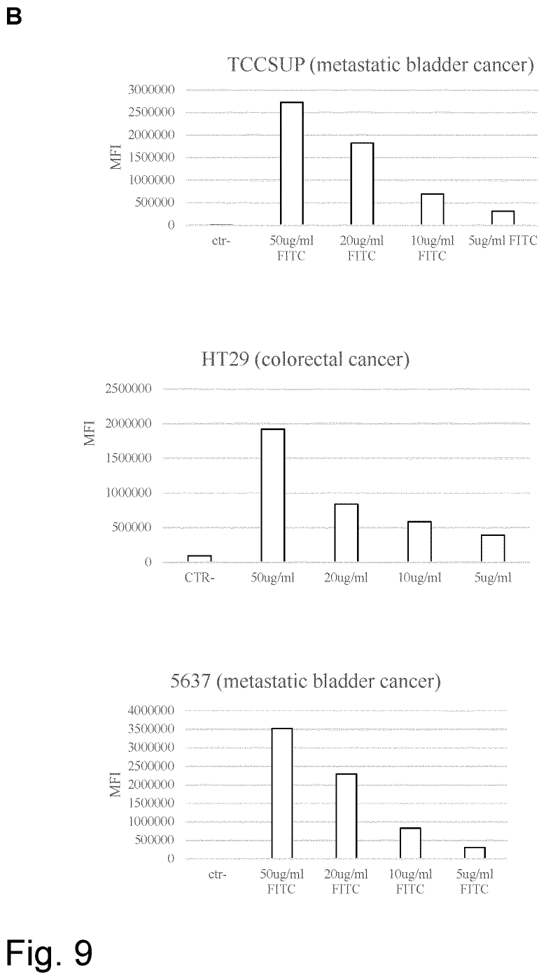

The selected DIA-12.3 IgG1 of the present invention was further characterized for the binding to different tumor cell lines by flow cytometry. The results reported on FIG. 9 showed a high percentage of positive cells and high MFI values at every concentration tested in two metastatic bladder cancer cells (TTCSUP and 5637 cell lines) and for the colorectal cancer cell line HT29.

### Example 4: LDH (Lactate Dehydrogenase) Cytotoxicity Assay

To evaluate the capability of the DIA-12.3 IgG1 antibody to affect the NK-92 cell cytotoxicity against tumor cells, the LDH-Glo™ Cytotoxicity Assays (Promega) was performed. This assay is a bioluminescence method that quantifies the enzymatic activity of LDH, a widely used marker of cytotoxicity, which is released in the cytoplasm upon cell membrane destruction.

Target tumor cells were harvested, washed, and resuspended at the final density of 2×10⁵ cells/mL in basic DMEM with 5% v/v of FBS. Target cells were incubated alone or with the DIA 12.3 antibody for 30 minutes at RT. Meanwhile, NK-92 cells were firstly diluted at 2×10⁶ cells/mL in basic RPMI medium with 5% v/v of FBS and then serially diluted ranging routinely from 1×10⁵ to 1×10³ cells. 50 μl/well of each NK-92 cell suspension dilution were seeded in 96 white walled assay plates, in triplicate. Following the antibody treatment, 50 μl/well of the target cell suspension were then added to the effector NK-92 cells at increasing effector/target cell ratio (E:T) ranging from 1:1 to 10:1 in triplicate. Positive controls were used by adding 2 μl/well of TritonX-100 to determine the maximum LDH release, while triplicate wells without cells were set up to serve as negative control to determine the culture medium background. The plate was incubated for 4 hours at 37° C. 5 μl of supernatants from each well were collected and diluted in 95 μl of LDH Storage buffer. The diluted samples were then stored at −20° C. and the day of the assay they were further diluted in the LDH Storage Buffer to fit the linear range of the assay. 50 μl of the diluted samples were transferred into a 96-well opaque, non-transparent assay plate. 50 μl of the LDH Detection Reagent were then added in each well and the plate was incubated at room temperature for 60 minutes. Luminescence was recorded from 30 to 60 minutes after the addition of the LDH reaction agent. The % of cytotoxicity was calculated as follows:

100×(Experimental LDH Release−Medium Background)/(Maximum LDH Release Control−Medium Background).

### Example 5: Flow Cytometry Assay on Effector Cells

DIA-12.3 IgG1 was evaluated for the binding of CEACAM1 expressed on the surface of neutrophil cells isolated from healthy donors. The analysis is carried out by dispensing 5×10⁵ neutrophils cells in 1 mL of medium per tube. The cells are then washed twice in PBS by centrifuging at 1,600 rpm for 6 min and finally resuspended in 200 μl of a blocking solution consisting of 1×PBS and 1% w/v BSA and incubated for 30 minutes at room temperature (RT). After this phase, the antibody diluted in 200 μl of block solution is added and incubated for 60 minutes at room temperature. At the end of the incubation, two washes are carried out in PBS and each cell pellet is resuspended in 200 μl of a solution consisting of an anti-human-FITC antibody diluted 1:1000 v/v in blocking solution, the incubation is maintained for 60 minutes at RT. Cells are then washed once with PBS and centrifuged as above, the supernatant is removed, and pellet resuspended in 500 μl of PBS. The samples were subjected to flow cytometric analysis with FacScan (Becton Dickinson). Neutrophils were reacted with 10 μg/ml of DIA-12.3 IgG1. Results are reported in FIG. 12.

The binding to CEACAM1 does not impact viability of effector cells. At this end, neutrophils isolated from 3 patients and enriched were incubated in RPMI medium in the presence or in the absence of DIA-12.3 IgG1 for 1, 2, 6, 18 and 20 h, at 37° C. At the end, the Annexin V/PI staining (Promega) has been performed, according to manufacturer's instructions. The results, shown in FIG. 13, do not show any difference between treated and untreated sample at any time points.

### Example 6

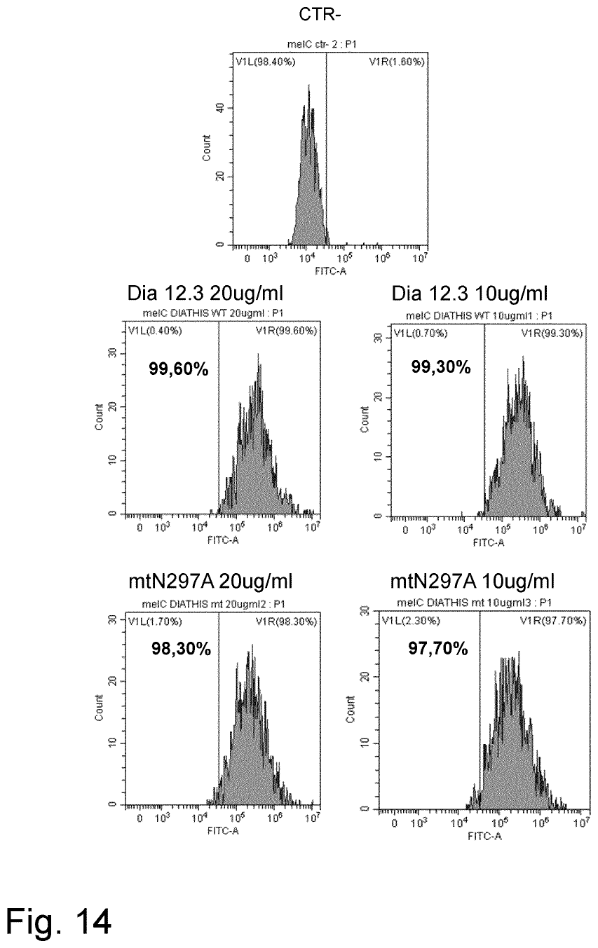

DIA-12.3 IgG1 and the mutated DIA-12.3 N297A IgG1 were tested for their reactivity on CEACAM1 expressed on melanoma cells by Flow cytometry. MelC cells were reacted with 20 or 10 μg/ml of DIA-12.3 IgG1 or with the IgG1 N297A mutated antibody. Negative control (CTR-) is represented by MelC cells reacted with secondary antibodies only. The N297A showed a reduced Fc effector functions, as highlighted in FIG. 14.

1. A fully human antibody specific for CEACAM1, comprising the light chain variable region sequences defined by SEQ ID NO: 1 SELTQDPAVSVALGQTVRITCQGDSLRSSYASWYRQRPGQAPVPVIYGKNNRPS GIPDRFSGSSSGNTASLTITGAQAEDEADYYCX6SSYAWLPYVVFGGGTKLTVLG wherein X6 is any naturally occurring amino acid; the heavy chain variable region sequences defined by SEQ ID NO: 2 and the human antibody Immunoglobulin G class I constant region sequences.

2. The antibody of claim 1, wherein X6 is N or Q or A or L.

3. The antibody of claim 1, wherein X6 is N.

4. The antibody of claim 1, wherein X6 is Q.

5. The antibody of claim 1, wherein the heavy chain constant region sequences are defined by SEQ ID NO: 7 ASTKGPSVFPLAPSSKSTSGGTAALGCLVKDYFPEPVTVSWNSGALTSGVHTFPA VLQSSGLYSLSSVVTVPSSSLGTQTYICNVNHKPSNTKVDKKVEPKSCDKTHTCP PCPAPELLGGPSVFLFPPKPKDTLMISRTPEVTCVVVDVSHEDPEVKFNWYVDGV EVHNAKTKPREEQYX5STYRVVSVLTVLHQDWLNGKEYKCKVSNKALPAPIEKT ISKAKGQPREPQVYTLPPSRDELTKNQVSLTCLVKGFYPSDIAVEWESNGQPENN YKTTPPVLDSDGSFFLYSKLTVDKSRWQQGNVFSCSVMHEALHNHYTQKSLSLS PGK, wherein X5 is any naturally occurring amino acid.

6. The antibody of claim 1, wherein the heavy chain constant region sequences are defined by SEQ ID NO: 9 ASTKGPSVFPLAPCSRSTSESTAALGCLVKDYFPEPVTVSWNSGALTSGVHTFPA VLQSSGLYSLSSVVTVPSSSLGTKTYTCNVDHKPSNTKVDKRVESKYGPPCPPCP APEFLGGPSVFLFPPKPKDTLMISRTPEVTCVVVDVSQEDPEVQFNWYVDGVEV HNAKTKPREEQFNSTYRVVSVLTVLHQDWLNGKEYKCKVSNKGLPSSIEKTISK AKGQPREPQVYTLPPSQEEMTKNQVSLTCLVKGFYPSDIAVEWESNGQPENNYK TTPPVLDSDGSFFLYSRLTVDKSRWQEGNVFSCSVMHEALHNHYTQKSLSLSLG K.

7. The antibody of claim 1, wherein the light chain constant region sequences are defined by SEQ ID NO: 8 GQPKAX1PX2VTLFPPSSEELQANKATLVCLISDFYPGAVTVAWKADX3SPVKAG VETTX4PSKQSNNKYAASSYLSLTPEQWKSHRSYSCQVTHEGSTVEKTVAPTECS, wherein X1, X2, X3, X4 are any naturally occurring amino acid.

8. The antibody of claim 5, wherein X5 is N or A.

9. The antibody of claim 7, wherein X1 is N or A, X2 is T or S, X3 is G or S, X4 is K or T.

10. The antibody of claim 1, wherein the light chain constant region sequences are defined by SEQ ID NO: 8 and the heavy chain constant region sequences are defined by SEQ ID NO: 7.

11. The antibody of claim 1, wherein the heavy chain constant region sequences are defined by SEQ ID NO: 4 and the light chain constant region sequences are defined by SEQ ID NO: 3.

12. The antibody of claim 1, wherein the heavy chain constant region sequences are defined by SEQ ID NO: 5 and the light chain constant region sequences are defined by SEQ ID NO: 3.

13. The antibody of claim 1, wherein the heavy chain constant region sequences are defined by SEQ ID NO: 4 and the light chain constant region sequences are defined by SEQ ID NO: 6.

14. The antibody of claim 1, wherein the heavy chain constant region sequences are defined by SEQ ID NO: 9 and the light chain constant region sequences are defined by SEQ ID NO: 3.

15. The antibody of claim 1, wherein said antibody is conjugated to at least one diagnostic and/or therapeutic agent to form an immunoconjugate.

16. The conjugated antibody of claim 15, wherein the therapeutic agent is selected from the group consisting of an antibody, an antigen-binding antibody fragment, a cytotoxic agent, a drug, a toxin, a radionuclide, boron atoms, an immunomodulator, a photoactive therapeutic agent, an immunoconjugate, an oligonucleotide and a hormone.

17. The conjugated antibody of claim 15, wherein the diagnostic agent is selected from the group consisting of a radionuclide, a radiological contrast agent, a paramagnetic ion, a metal, a fluorescent label, a chemiluminescent label, an ultrasound contrast agent and a photoactive agent.

18. A pharmaceutical composition comprising an antibody according to claim 1.

19. The composition of claim 18, further comprising one or more therapeutic agents.

20. The composition according to claim 18, for use in a method for the treatment of medullary thyroid cancer (MTC), colorectal cancer, hepatocellular carcinoma, liver cancer, gastric cancer, oesophageal cancer, lung cancer, non-small cell lung cancer, breast cancer, pancreatic cancer, ovarian cancer, uterine cancer, cervical cancer, endometrial cancer, head-and-neck cancer, bladder cancer, urothelial cancer, prostate cancer, hematopoietic cancer, leukaemia, or melanoma.

21. The composition according to claim 18, for use in a method for the diagnosis of cancer.

22. The composition for use according to claim 20, wherein said composition is used in combination with at least one diagnostic and/or therapeutic agent.
